(19) United States
(12) Patent Application Publication
(10) Pub. No.: US 2023/0149059 A1
(43) Pub. Date: May 18, 2023

(54) FLEXIBLE INTRAMEDULLARY NAIL

(71) Applicant: Conventus Orthopaedics, Inc., Maple Grove, MN (US)

(72) Inventors: Kevin Rzasa, Wilmington, DE (US); Jason Cianfrani, Conshohocken, PA (US); Tim Schmucker, Glenolden, PA (US)

(21) Appl. No.: 17/525,075

(22) Filed: Nov. 12, 2021

(51) Int. Cl. A61B 17/72

(52) U.S. Cl. CPC A61B 17/7208; A61B 17/7241

(57) ABSTRACT

A surgical nail, such as an intramedullary nail, includes a nail body that includes a proximal end, an elongate intermediate portion comprising an intermediate flexible portion; and a distal end. The proximal end and the distal end are coupled and offset from one another by the elongate intermediate portion. The wherein the intermediate flexible portion comprises at least two cables boned one to another and configured to maintain a fixed length. The at least one cable comprises two or more cables twisted around one another in a helical arrangement and welded together. The proximal portion and the threaded end portion can be coupled together by the intermediate flexible portion as a unitary, integrated element prior to any use of the surgical nail in an implant procedure. The intermediate flexible portion is configured to permit the proximal portion to bend at an angle relative to the distal portion as a unitary, integrated element.

### TECHNICAL FIELD

The present teachings relate to intramedullary (IM) fixation. More particularly, the present teachings relate to an implant and a method for implanting an implant in a medullary canal in performing intramedullary fixation.

### BACKGROUND

Surgical procedures to repair bone fractures can include the use of implants, such as plate fixation, IM nails, and interfragmentary screws, that are commonly associated with complications such as infection, wound breakdown, nonunion, implant failures, poor cosmetic outcome, and local numbness, etc. The term “intramedullary” means that the nail resides at least partly in the medullary canal of a bone. IM fixation involves the treatment of unstable fractures with an intramedullary nail as a treatment option for bone fractures and other injuries. Generally, intramedullary fixation devices for bone fractures are complicated by the need to perform reliable fixation of the bone while providing some flexibility supporting anchoring and/or improving fixation of the device. Additionally, “interfragmentary” screws are used to provide compression between the fracture fragments to stabilize the fracture.

In one example, U.S. Pat. No. 7,625,395 to Helmut Muckter (“Muckter”) discloses an interfragmentary screw that is required to be implemented in separate pieces during implantation. For example, Muckter discloses that a threaded part with a bone thread must be screwed into the bone utilizing a cannulated wrench that is pushed over a wire cable before a hexagon socket head nut is subsequently attached with a metal thread. Additionally, Muckter’s interfragmentary screw may not be utilizable in procedures that require minimizing bone compression.

Improvements in IM fixation are therefore desired.

### BRIEF SUMMARY OF THE DISCLOSED EMBODIMENTS

During the preparation and placement of existing intramedullary nails and associated syndesmotic fixation, there is the potential for the placement of those syndesmotic members be overly rigid and inflexible, complicating the healing process and introducing instability to the fixation members. Additionally, some known syndesmotic members can be configured in a way that introduces undesirable bone compression in certain injuries. This is solved in the presently disclosed embodiments by providing a surgical nail that limits bone compression yet imparts flexibility to an implant and therefore the healing bone, according to some embodiments.

According to embodiments, a surgical nail can include a nail body having a proximal end, an elongate intermediate portion comprising an intermediate flexible portion, and a distal end. In some embodiments, the proximal end and the distal end are coupled and offset from one another by the elongate intermediate portion. The intermediate flexible portion can include two or more cables that are bonded together (e.g., by welding, adhesive bonding, fusing, and/or the like) to maintain a fixed length.

According to some embodiments, the cable can include two or more cables twisted around one another in a helical arrangement and bonded together. In some embodiments, the proximal portion can include a threaded proximal portion coupled to the threaded portion by the intermediate flexible portion. According to some embodiments, the distal end portion can include a threaded end portion.

According to some embodiments, the proximal portion can include a threaded proximal portion coupled to the threaded end portion by the intermediate flexible portion. In some embodiments, the proximal end portion can include a cylinder having an outer surface defining a perimeter of the surgical nail.

According to some embodiments, the one or more cables is configured in one or more arrangements consisting of a Helical Hollow Strand (HHS) arrangement, and/or a simple stranded cable arrangement. In some embodiments, the proximal portion and the threaded end portion are coupled together by the intermediate flexible portion as a unitary, integrated element prior to any use of the surgical nail in an implant procedure. According to some embodiments, the cylinder can include at least one through holes for receiving a fixation element configured to anchor the surgical nail into a bone fragment.

According to some embodiments, the intermediate flexible portion is configured to permit the proximal portion to bend at an angle relative to the distal portion such that a health practitioner is enabled to implant the surgical nail in a medullary canal as a unitary, integrated element. Also, in some embodiments, the intermediate flexible portion is configured to be flexible when implanted in the medullary canal and is further configured to minimize bone compression.

A method for performing implantation of a surgical nail during a surgical procedure to repair a fracture of a bone is described. According to some embodiments the method can include identifying a starting point of a medullary canal of a patient’s bone; providing an opening in the bone using a surgical device; inserting the surgical nail as a unitary element into the medullary canal; driving the surgical nail through a first bone fragment via a portion of the medullary canal; and fixing the intramedullary canal to a second bone fragment, where the intramedullary nail is be flexibly fixed to the bone first and second bone fragments and is configured to minimize compression of the bone. The intermediate flexible portion can be configured to be flexible when implanted in the medullary canal and is further configured to minimize bone compression.

### BRIEF DESCRIPTION OF THE DRAWINGS

FIG. 1 is an illustration of a surgical nail for use in syndesmotic fixation procedures, according to some embodiments.

FIG. 2 is an illustration of a surgical nail for use in syndesmotic fixation procedures, according to some embodiments.

FIG. 3 is an illustration of a surgical nail for use in syndesmotic fixation procedures, according to some embodiments.

FIG. 4 is an illustration of a surgical nail for use in syndesmotic fixation procedures, according to some embodiments.

FIG. 5A depicts a tool for implanting a surgical nail for use in syndesmotic fixation procedures, according to some embodiments.

FIG. 5B depicts a surgical nail implanting system for use in syndesmotic fixation procedures, according to some embodiments.

FIG. 6 depicts an exemplary surgical nail implantation in a surgical procedure to heal a clavicle, according to some embodiments.

FIG. 7 is a flow diagram of a method for performing an implantation procedure of an intramedullary nail having a flexible intermediate portion, according to some embodiments.

### DETAILED DESCRIPTION OF THE DISCLOSED EMBODIMENTS

Embodiments may be implemented in hardware, firmware, software, or any combination thereof. Intramedullary fixation can be performed utilizing surgical nails, such as intramedullary nails, to facilitate the healing of fractured bones. However, rigid intramedullary nails that do not sufficiently flex can impede anchoring to bone fractures and aggravate the healing process. Further, conventional intramedullary nails having some degree of flexibility may compress and cause additional complications. The embodiments shown in the exemplary methods and devices are not exhaustive and other operations can be performed in addition to the illustrated processes. In some embodiments of the present disclosure, the operations may vary and/or can be performed in a different order.

### Surgical Nail with Flexible Portion

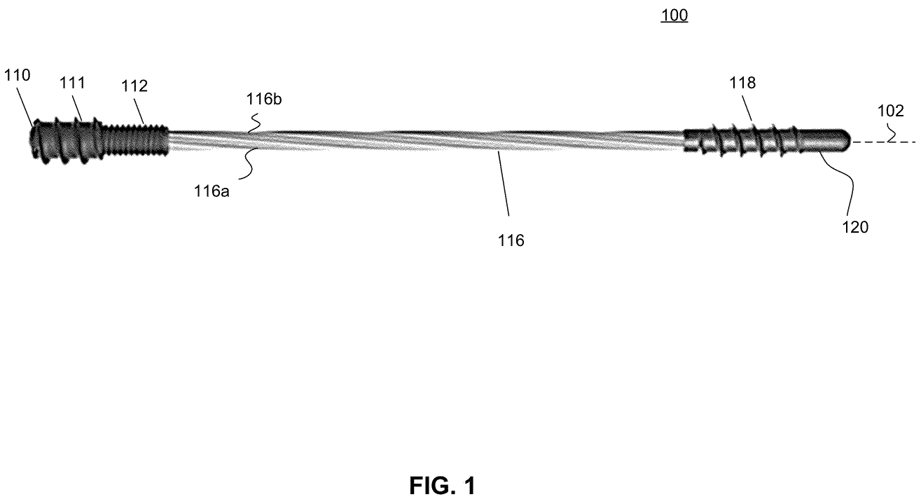

FIG. 1 illustrates a surgical nail 100, which may be an intramedullary nail, that is configured to be flexible and support bone fragments during a bone healing process. Surgical nail 100 includes a nail body that can be elongate along a central axis 102. The nail body can comprise a proximal end 110, an elongate intermediate portion comprising an intermediate flexible portion 116, wherein the intermediate flexible portion comprises two or more cables that are bonded together (e.g., by welding, adhesive bonding, fusing, and/or the like) to maintain a fixed length; and a distal end 120. As shown, proximal end 110 and distal end 120 are coupled and offset from one another by the elongate intermediate portion 116.

According to some embodiments, intermediate flexible portion 116 is configured to be bendable throughout its length. According to some embodiments, intermediate flexible portion 116 is configured to resist compression. In this regard, these embodiments differ substantially from concepts related to interfragmentary screws that may be configured to achieve compression. For example, in the embodiment illustrated in FIG. 1, intermediate flexible portion 116 is configured by bonded cables 116a and 116b to resist compression, which is distinct and different Muckter’s interfragmentary screw that is configured to provide bone compression. Instead, surgical nail 100 is configured by intermediate flexible portion 116 to be flexible without introducing bone compression.

In some embodiments, intermediate flexible portion 116 can comprise two or more cables 116a and 116b bonded together. For example, two or more cables 116a and 116b can be twisted and bonded together in a helical arrangement. In other embodiments, two or more cables 116a and 116b can be welded together in a braided arrangement.

In some non-limiting examples, cable 116 can be a single or multi-layered Helical Hollow Strand (HHS) tube. In another example, cable 116 can be a simple stranded cable arranged in various n×m cable classifications, where n represents the number of strands in a cable and m represents the number of wires in each strand (e.g., 1×19, 1×7, 7×19, etc.). In some examples, cable 116 can be a multi-layered multi-directional cable. Additionally, cable 116 can be solid or cannulated.

In one non-limiting example, the component bodies such as proximal end 110, intermediate flexible portion 116, and distal end 120, can be bonded together (e.g., by welding, adhesive bonding, or otherwise joining) at a fixed length prior to the point of use. In this integrated implementation, surgical nail 100 is configured to be inserted in a medullary canal as a unitary structure, not to be inserted as independent component bodies as in prior art devices. According to some embodiments, the intermediate flexible portion is configured to permit the proximal end to bend at an angle relative to the distal end such that the health practitioner is enabled to implant the surgical nail in a medullary canal as a unitary, integrated element. For example, surgical nail 100 can be joined stably by bonding each component to another, such that the whole assembly rotates as one.

According to some embodiments, proximal end 110 includes one or more threaded portions. As shown, for example, proximal end 110 can include first proximal threaded portion 111 and second proximal threaded portion 112. Also, as shown, distal end 120 of surgical nail 100 can include threaded end portion 118. Each of the threaded portions 111, 112, and 118 can be configured having cutting threads capable of being driven into one or more bone fragments. For example, threaded end portion 118 can be driven, for example, by a driving device (as described hereinbelow) such that threaded end portion 118 is fixed into a bone fragment. Likewise, threaded proximal portion 111 and threaded intermediate portion 112 can be driven to fix the cutting threads into corresponding portions of a bone fragment proximal to an opening lumen in the bone. Also, according to some embodiments (not shown) a surgical nail can be configured to perform proximal or distal end fixation, e.g., by an anchoring element.

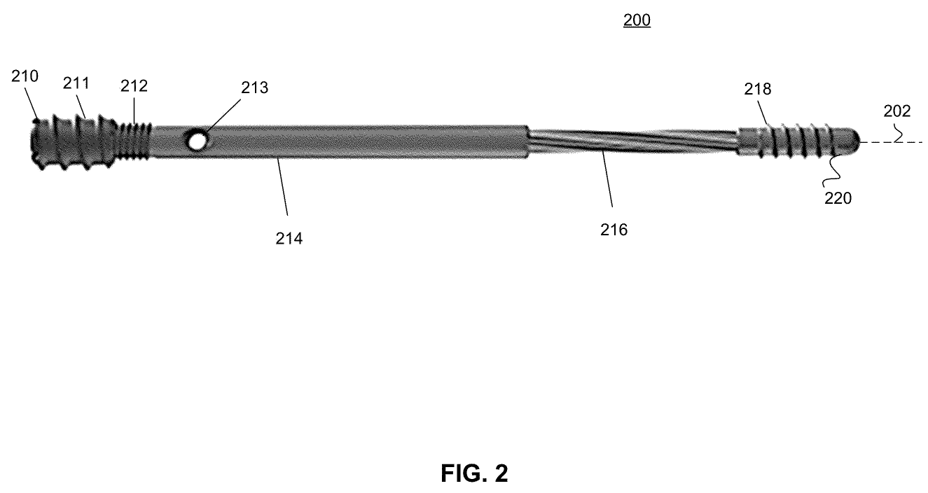

FIG. 2 illustrates a surgical nail 200, which may be an intramedullary nail, that is configured to be flexible and support bone fragments during a bone healing process. Surgical nail 200 includes a nail body that can be elongate along a central axis 202. The nail body can comprise a proximal end 210 having a first proximal threaded portion 211, a second proximal threaded portion 212, an elongate intermediate portion comprising an intermediate flexible portion 216, wherein the intermediate flexible portion comprises two or more cables that are bonded together (e.g., by welding, adhesive bonding, fusing, and/or the like) to maintain a fixed length, and a distal end 220. Surgical nail 200, which may be an embodiment of surgical nail 100, can include proximal end 210 and distal end 220 coupled to and offset from one another by the elongate intermediate portion 216.

As above, intermediate flexible portion 216 is configured to be bendable throughout its length. According to some embodiments, intermediate flexible portion 216 is configured to resist compression. In this regard, these embodiments differ substantially from concepts related to interfragmentary screws that may be configured to achieve compression. In one example, cables 216a and 216b can be formed of any flexible material, such as metal and/or metal alloy material in some embodiments. For example, cables 216a and 216b can be formed of steel, iron, aluminum, copper, nickel, any other suitable metal material, fiber, metal-fiber, polymer, and/or any other flexible material. Cables 216a and 216b can be welded (or otherwise bonded) to one another to avoid unraveling and to improve stability of the cables. Additionally, as described above, bonding cables 216a and 216b configures intermediate flexible portion 216 to resist compression.

According to some embodiments, proximal end 210 includes one or more threaded portions. As shown, for example, proximal end 210 can include first proximal threaded portion 211 and second proximal threaded portion 212. According to some embodiments, distal end 220 can include threaded end portion 218.

According to additional embodiments, surgical nail 200 can include a cylindrical body portion 214 and at least one through hole 213 for receiving a fixation element configured to anchor the surgical nail into a bone fragment (not shown). Cylindrical body portion 214 defines an outer perimeter of surgical nail 200 and is disposed having at least one through hole 213 entering one side of the surgical nail outer perimeter and exiting through the other side of the outer perimeter. In this manner, surgical nail 200 is configured to accept transverse screws for fixation of surgical nail 200 to one or more bone fractures.

In some embodiments, intermediate flexible portion 216 comprises two or more cables 216a and 216b bonded together. For example, cables 216a and 216b can be twisted and bonded together in a helical arrangement. In other embodiments, cables 216a and 216b can be bonded together in a braided arrangement.

In some non-limiting examples, cable 216 can be a single or multi-layered HHS tube, or a simple stranded cable arranged in various n×m cable classifications, where n represents the number of strands in a cable and m represents the number of wires in each strand as described in detail above, a multi-layered multi-directional cable, and/or any other arrangement of a cable. Additionally, cable 216 can be solid or cannulated.

As noted above, surgical nail 200 can include one or more threaded portions, such as threaded proximal portion 211, threaded intermediate portion 212 and threaded end portion 218. Each of the threaded portions, 211, 212, and 218 can be configured using cutting threads to be driven into one or more bone fragments. For example, following insertion through a medullary canal, threaded end portion 218 can be driven by a driving device (as discussed hereinbelow) such that threaded end portion 218 is fixed into a bone fragment. Likewise, threaded proximal portion 211 and threaded intermediate portion 212 can be driven to fix the cutting threads into corresponding portions of a bone fragment proximal to an opening lumen in the bone.

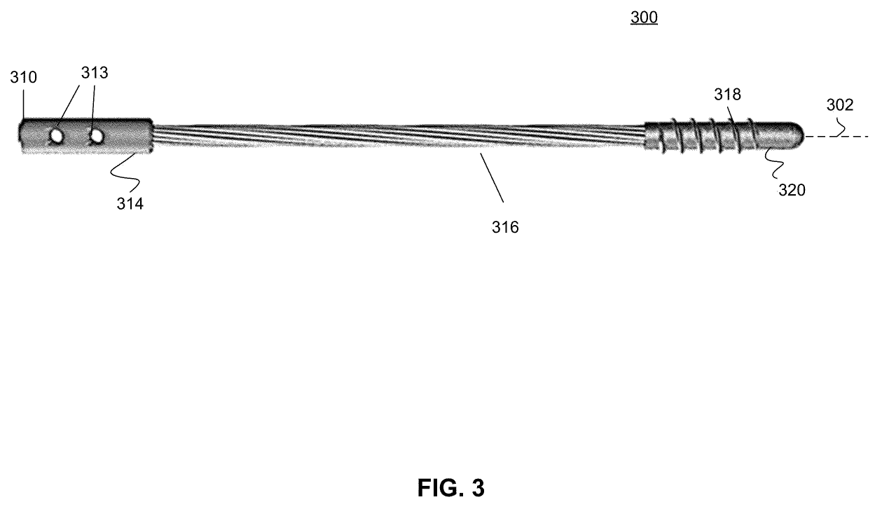

FIG. 3 illustrates a surgical nail 300, which may be an intramedullary nail, that is configured to be flexible and support bone fragments during a bone healing process. Surgical nail 300 can be an embodiment of surgical nail 100 and/or 200. Surgical nail 300 can include a nail body that can be elongate along a central axis 302. The nail body can comprise a proximal end 310, an elongate intermediate portion comprising an intermediate flexible portion 316, wherein the intermediate flexible portion comprises two or more cables that are bonded together (e.g., by welding, adhesive bonding, fusing, or the like) to maintain a fixed length, and a distal end 320. As shown, proximal end 310 and distal end 320 are coupled and offset from one another by the elongate intermediate portion 316.

According to some embodiments, the intermediate flexible portion 316 is configured to be bendable throughout its length. As above, intermediate flexible portion 316 is configured to resist compression as opposed to interfragmentary screws that may be configured to achieve compression.

According to some embodiments, surgical nail 300 can include a cylindrical body portion 314 and at least one through hole 313 for receiving a fixation element configured to anchor the surgical nail into a bone fragment. For example, after implantation of surgical nail in a surgical procedure, a health practitioner can drive one or more screws into through holes 313 to fix and anchor a first fragment of the bone to surgical nail 300, such that it is retained sufficiently to heal together with a second fragment of the bone.

As above, intermediate flexible portion 316 is flexible and configured by two or more cables 316a and 316b to be bendable without producing compression. By permitting flexibility without compression, intermediate flexible portion 316 allows the surgical nail to stay stably fixed to each bone fragment minimizing risks of nonunion or aggravation to the healing process. Cables 316a and 316b can be formed of any flexible material, such as one or more metal and/or metal alloy material. Cables 316a and 316b can be bonded (such as by welding, adhesives, or any other suitable bonding process or device) one cable to another to another to avoid unraveling and to improve stability of the cables. In some embodiments, a ratio of length l_{c} of cylindrical body portion 314 to length lᵢ of flexible intermediate portion 316 may be large relative to a corresponding ratio in the embodiment of FIG. 2. Such an implementation may be suitable in cases where a greater degree of flexibility is desired, while maintaining resistance to bone compression.

As described in detail above, cable 316 can be a single or multi-layered HHS tube, or a simple stranded cable arranged in various n×m cable classifications, where n represents the number of strands in a cable and m represents the number of wires in each strand, a multi-layered multi-directional cable, and/or any other arrangement of a cable. Additionally, cable 316 can be solid or cannulated.

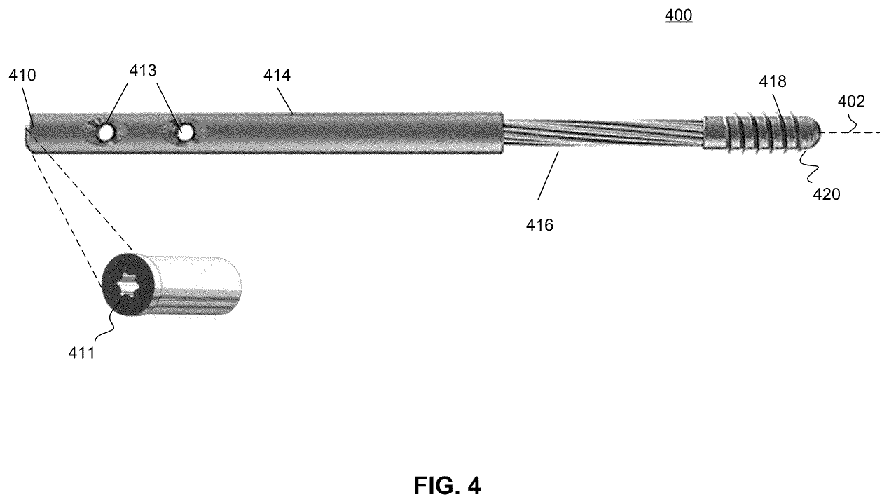

FIG. 4 illustrates a surgical nail 400, which may be an intramedullary nail, that is configured to be flexible and support bone fragments during a bone healing process. Surgical nail 400 includes a nail body that can be elongate along a central axis 402. The nail body can comprise a proximal end 410, an elongate intermediate portion comprising an intermediate flexible portion 416, wherein the intermediate flexible portion comprises two or more cables that are bonded together (e.g., by welding, adhesive bonding, fusing, or the like) to maintain a fixed length, ;and a distal end 420. As shown, proximal end 410 and distal end 420 are coupled and offset from one another by the elongate intermediate portion 416.

According to some embodiments, proximal end 410 can include a drive socket 411 to receive and engage a driver of an implement useful to insert surgical nail 400 into a medullary canal. For example, drive socket 411 of intramedullary nail 400 may include a Hexalobe opening therein that can be rotated by a driver inserted therein to cause intramedullary nail 400 to be inserted as a unitary element into a medullary canal of a bone.

As in the embodiment of FIG. 3, a ratio of length l_{c} of cylindrical body portion 314 to length lᵢ of flexible intermediate portion 316 can be adapted to achieve an intended degree of flexibility and/or anchor support area. In this embodiment, the ratio is small relative to a corresponding ratio in the embodiment of FIG. 3, which may permit greater rigidity in specific bone healing processes utilizing surgical nail 400.

As above, the intermediate flexible portion 416 is flexible and may be configured to bend. According to some embodiments, intermediate flexible portion 416 is configured to resist compression. In this regard, these embodiments differ substantially from concepts related to interfragmentary screws that may be configured to achieve compression. For example, intermediate flexible portion 416 is configured by two or more bonded cables 416a and 416b to resist compression, which is distinct and different Muckter’s interfragmentary screw that is configured to achieve bone compression.

In some embodiments, intermediate flexible portion 416 can comprise two or more cables 416a and 416b bonded together. For example, cables 416a and 416b can be twisted and bonded together in a helical arrangement. In other embodiments, cables 416a and 416b can be bonded together in a braided arrangement.

According to additional embodiments, surgical nail 400 can include a cylindrical body portion 414 and at least one through hole 413 for receiving a fixation element configured to anchor the surgical nail into a bone fragment (not shown).

In some non-limiting examples, cable 416 can be a single or multi-layered HHS tube, a simple stranded cable arranged in various n×m cable classifications, as described above. In some examples, cable 416 can be a multi-layered multi-directional cable. Additionally, cable 416 can be solid or cannulated.

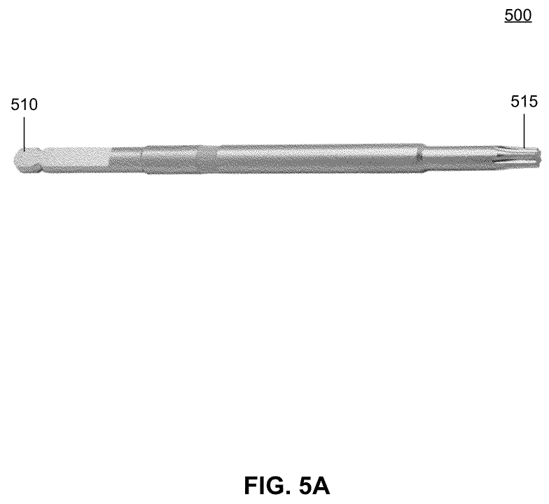

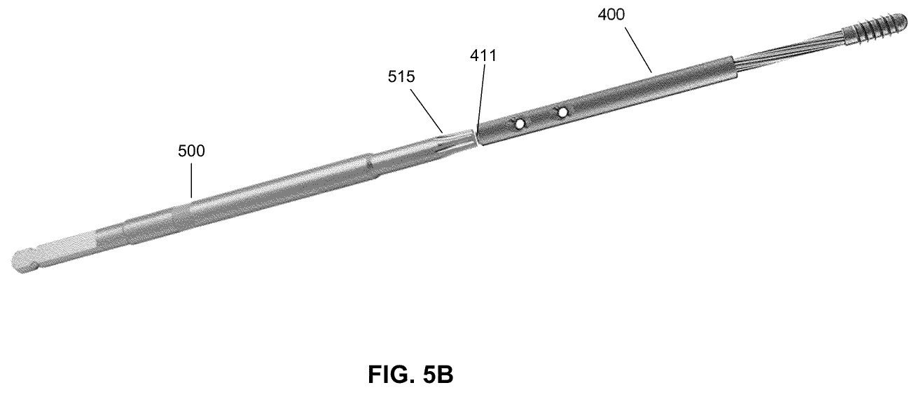

FIG. 5A illustrates an exemplary driving device 500 for use in inserting a surgical nail, according to some embodiments. Driving device 500 can include a tang 510 and a driver 515 configured to engage the drive socket at the proximal end of the surgical nail. Driving device 500 is illustrated as one example of a device to engage a surgical nail, such as an intramedullary nail according to embodiments of any of FIGS. 1 to 4. However, any suitable device may be utilized to implant a surgical nail. As shown, exemplary driving device 500 can include driver 515, which may include a solid or cannulated Hexalobe driver, in one example.

FIG. 5B illustrates an embodiment of an intramedullary nail system that can include driving device 500 that can be utilized to insert a surgical nail, such as intramedullary nail 400. As shown, driver 515 can be a Hexalobe driver configured to engage a Hexalobe drive socket 411 of intramedullary nail 400. The driving device can be rotated to insert intramedullary nail 400 as a unitary element into a medullary canal of a bone.

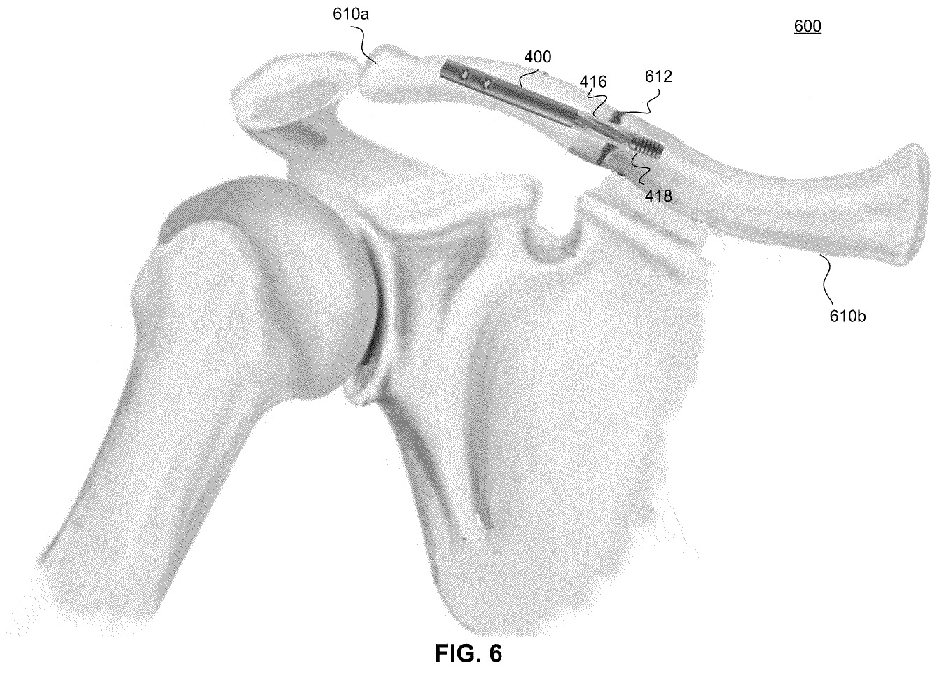

In one non-limiting example, as shown in FIG. 6, intramedullary nail 400 can be inserted in a clavicle 610 (e.g., a first bone fragment 610a) of a patient, where clavicle 610 has suffered a fracture 612. Driving device 500 can be configured to drive the unitary intramedullary nail such that a threaded end portion 418 is fixed into a second bone fragment 610b and a proximal portion 410 having at least one through hole can be fixed by at least one anchoring element (not shown) into the bone fragment 610a. The above embodiment is illustrated as one non-limiting example of system that includes a surgical nail, such as an intramedullary nail according to embodiments of any of FIGS. 1 to 4 having a flexible intermediate portion, and a device to engage the surgical nail.

### Performing Intramedullary (IM) Fixation Using Intramedullary Nail Having Bonded Cables

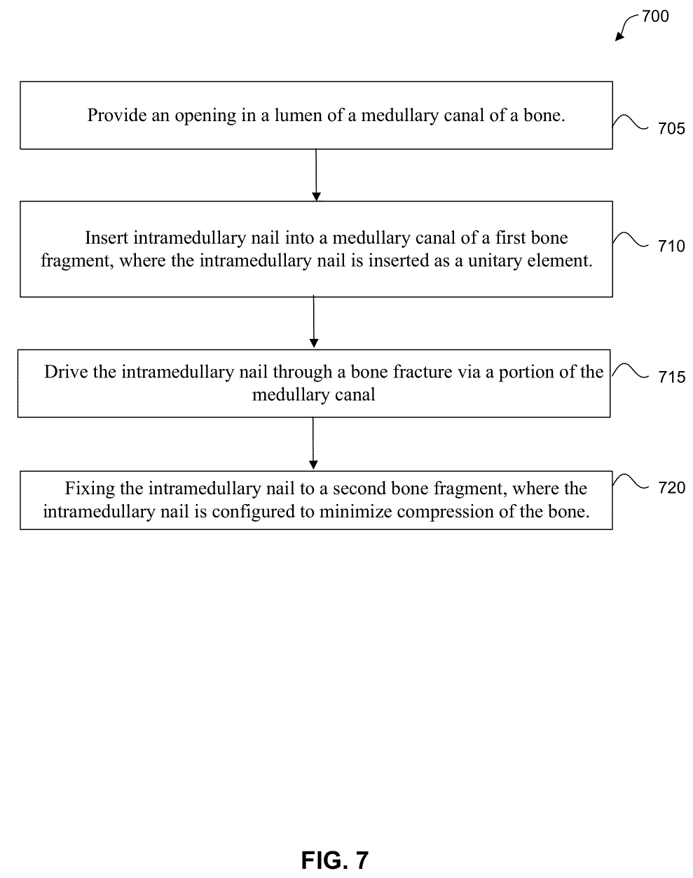

FIG. 7 depicts a flow diagram of a method for performing a surgical procedure 700 to drive an intramedullary nail having bonded cables (e.g., for repair of clavicle, ribs, etc.) that impart flexibility to an implant and therefore the healing bone, according to some embodiments.

Referring to FIG. 7, surgical procedure 700 includes an operation 705 of identifying a starting point on an end, such as a distal end, of a bone and creating an opening in a lumen of a medullary canal of the bone. For example, the starting point can be confirmed by a user of surgical nail 400 (e.g., a health care practitioner, or the like). The starting point may be on a distal tip of the bone in some embodiments. In other embodiments, the starting point may be on a proximal end. In some embodiments, operation 705 can include insertion of a guide wire from the tip into the intramedullary canal.

A surgical nail 400 is discussed for illustration, although any method 700 can be implemented using any embodiment of a surgical nail (e.g., 100, 200, 300, 400). According to some embodiments, a reamer or other suitable device can be used to access a lumen of the bone. For example, a patient may be prepared for surgery, including placing the patient under general anesthesia or sedation, administering antibiotics, and placing the patient on an operating room table. A radiographic/fluoroscopic imaging device can be directed toward the site of the procedure. According to some embodiments, reaming can be performed.

Procedure 700 continues with operation 710, in which the health practitioner can insert the surgical nail 400 into the medullary canal of a first bone fragment, where the surgical nail is inserted as a unitary element. In other words, the surgical nail is disposed such that the proximal end 410 and distal end 420 are integrated together by the intermediate flexible portion 416 prior to use/insertion into the medullary canal. According to some embodiments, the intermediate flexible portion is configured to permit the proximal end to bend at an angle relative to the distal end such that the health practitioner is enabled to implant the surgical nail in a medullary canal as a unitary, integrated element.

Procedure 700 continues with operation 715, in which the health practitioner can driver the intramedullary nail through a bone fracture via a portion of the medullary canal. According to some embodiments, the unitary surgical nail 400 is inserted utilizing a driving device, such as driving device 500.

Procedure 700 continues with operation 720, where the health practitioner fixes the surgical nail to a second bone fragment. In some examples, a threaded end portion 418 is driven utilizing driving device 500 into the second bone fragment. In an embodiment, syndesmotic fixation members can be placed, for example, in through holes 413 to minimize enable the bone fragments to join efficiently having some degree of flexibility while minimizing compression of the bone.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments but should be defined only in accordance with the following claims and their equivalents.

1. A surgical nail comprising:
a nail body comprising:
a proximal end,
an elongate intermediate portion comprising an intermediate flexible portion; and
a distal end, wherein the proximal end and the distal end are coupled and offset from one another by the elongate intermediate portion, and
wherein the intermediate flexible portion comprises at least two cables bonded one to another and configured to maintain a fixed length.

2. The surgical nail of claim 1, wherein the at least one cable comprises two or more cables twisted around one another in a helical arrangement and welded together.

3. The surgical nail of claim 1, wherein the proximal portion comprises a threaded proximal portion coupled to the distal end by the intermediate flexible portion.

4. The surgical nail of claim 1, wherein the distal end comprises a threaded end portion.

5. The surgical nail of claim 4, wherein the proximal portion comprises a threaded proximal portion coupled to the threaded end portion by the intermediate flexible portion.

6. The surgical nail of claim 1, wherein the proximal end portion comprises a cylinder having an outer surface defining a perimeter of the surgical nail.

7. The surgical nail of claim 1, wherein the one or more cables is configured in one or more arrangements consisting of a Helical Hollow Strand (HHS) arrangement, a simple stranded cable arrangement and/or a multi-layered-multidirectional arrangement.

8. The surgical nail of claim 1, wherein the proximal end and the distal end are coupled together by the intermediate flexible portion as a unitary, integrated element prior to any use of the surgical nail in an implant procedure,
wherein the intermediate flexible portion is configured to permit the proximal end to bend at an angle relative to the distal end such that a health practitioner is enabled to implant the surgical nail in a medullary canal as a unitary, integrated element.

9. The surgical nail of claim 8, wherein the intermediate flexible portion is configured to be flexible when implanted in the medullary canal and is further configured to minimize bone compression.

10. The surgical nail of claim 6, wherein the cylinder comprises at least one through hole for receiving a fixation element configured to anchor the surgical nail into a bone fragment.

11. A method for performing implantation of a surgical nail during a surgical procedure to repair a fracture of a bone, the method comprising:
identifying a starting point of a medullary canal of a patient’s bone;
providing an opening in the bone using a surgical device;
inserting the surgical nail as a unitary element into the medullary canal;
driving the surgical nail through a first bone fragment via a portion of the medullary canal; and
fixing the intramedullary canal to a second bone fragment, wherein at least a portion of the intramedullary nail is flexible between the first and second bone fragments and is configured to minimize compression of the bone.

12. The method of claim 11, wherein the surgical nail comprises a nail body comprising:
a proximal end,
an elongate intermediate portion comprising an intermediate flexible portion; and
a distal end, wherein the proximal end and the distal end are coupled and offset from one another by the elongate intermediate portion, and
wherein the intermediate flexible portion comprises at least two cables bonded to one to another and configured to maintain a fixed length.

13. The method of claim 12, wherein the at least one cable comprises two or more cables twisted around one another in a helical arrangement and welded together.

14. The method of claim 12, wherein the proximal portion comprises a threaded proximal portion coupled to the threaded portion by the intermediate flexible portion.

15. The method of claim 12, wherein the distal end portion comprises a threaded end portion.

16. The method of claim 15, wherein the proximal portion comprises a threaded proximal portion coupled to the threaded end portion by the intermediate flexible portion.

17. The method of claim 12, wherein the proximal end portion comprises a cylinder having an outer surface defining a perimeter of the surgical nail.

18. The method of claim 12, wherein the one or more cables is configured in one or more arrangements consisting of a Helical Hollow Strand (HHS) arrangement, a simple stranded cable arrangement, and/or a multi-layered-multidirectional arrangement.

19. The method of claim 12, wherein the intermediate flexible portion is configured to be flexible when implanted in the medullary canal and is further configured to minimize bone compression.

20. An intramedullary nail system operable to repair a bone fracture during a surgical procedure to implant a surgical component, the intramedullary nail system comprising:
an intramedullary nail comprising a nail body comprising:
a proximal end comprising a drive socket;
an elongate intermediate portion comprising an intermediate flexible portion; and
a distal end, wherein the proximal end and the distal end are coupled and offset from one another by the elongate intermediate portion, and
wherein the intermediate flexible portion comprises at least one cable bonded to the proximal end and the distal end at a fixed length.
a driving device comprising a driver configured to engage the drive socket, wherein the driving device is a solid or cannulated Hexalobe driver.
